(19) United States
(12) Patent Application Publication
(10) Pub. No.: US 2025/0131188 A1
(43) Pub. Date: Apr. 24, 2025

(54) SYSTEMS AND METHODS FOR AUTOMATED CLINICAL DOCUMENT GENERATION

(71) Applicant: Synterex, Inc., Dedham, MA (US)

(72) Inventors: Jeanette M. Towles, Dedham, MA (US); Jonathan J. Towles, Dedham, MA (US); Jason Casavant, Dracut, MA (US); Tushar Goswami, Panchkula, IN (US); Shivam Goswami, Panchkula, IN (US); Sadkirat Singh, Punjab (IN)

(21) Appl. No.: 18/906,062

(22) Filed: Oct. 3, 2024

Related U.S. Application Data
(60) Provisional application No. 63/542,116, filed on Oct. 3, 2023.

(51) Int. Cl. G06F 40/186; G06F 40/40; G16H 15/00

(52) U.S. Cl. CPC G06F 40/186; G06F 40/40; G16H 15/00

(57) ABSTRACT

A software system simplifies and expedites the generation of patient-facing documents and other essential documents used in clinical trials. Through the integration of AI models, a biochemistry-oriented knowledge base, and Language Model Learning (LLM) mechanisms, the software system is capable of quickly producing documents that balance technical correctness with readability, thus substantially enhancing the productivity of clinical trial initiation and management processes. The software system operates through a sophisticated pipeline that ingests clinical trial protocol documents as inputs and processes them to generate comprehensive patient-facing documents along with other vital documents needed for clinical trials. This procedure, scalable and adaptive, consists of several nuanced stages.

### CROSS-REFERENCES

This application claims priority of U.S. provisional application Ser. No. 63/542,116 filed Oct. 3, 2023 and titled “SYSTEMS AND METHODS FOR AUTOMATED CLINICAL DOCUMENT GENERATION” by the present inventors.

### BACKGROUND

A clinical trial is a research study having the purpose of determining new or better ways to prevent, detect, or treat health conditions. Clinical trials are conducted, for example, to study whether a new test, treatment or preventive measure is safe and effective. Tests can include ways to screen for, diagnose, or prevent a disease or condition. Treatments and preventive measures can include medications, surgeries, medical devices, and behavioral therapies.

Government regulations and best practice methodologies require informed consent from clinical trial participants. Informed consent typically involves the elements of disclosing to potential research subjects the information needed to make an informed decision, facilitating the understanding of what has been disclosed, and promoting the voluntariness of the decision to participate or not in the clinical trial.

Patient-facing documents are an important part of informed consent. The documents include both information documents and agreements to be executed by the clinical trial participant. Conventionally, human writers having medical and science expertise have been employed to craft patient-facing documents for clinical trials. Generally, producing patient-facing documents involves a high level of technical expertise in science, medicine, pharmaceuticals, chemistry, government regulation and organization policy as well as a high-level skill in attention to detail. The patient-facing documents typically also need to meet government regulations and organization requirements. Finally, the language used in the documents needs to be understandable by the potential participants who are typically unskilled in science, medicine, etc.

It remains desirable to have improved methods and systems for generating clinical documents.

### SUMMARY

Embodiments of the present invention include systems and methods for generating patient-facing documents and other essential documents needed in clinical trials. The systems and methods enable simplified and expedited generation of patient-facing documents and other essential documents pivotal in clinical trials. At least one embodiment includes the integration of AI models, a biochemistry-oriented knowledge base, and Language Model Learning (LLM) mechanisms. Embodiments of the document generation system are capable of quickly producing documents that balance technical correctness with readability, having the benefits of substantially enhancing the productivity of clinical trial initiation and management processes.

Embodiments of the system are designed with the flexibility to broaden functionalities, enabling the creation of other clinical trial documents including trial protocols, investigator brochures, study reports, and regulatory submissions, which are integral components in the clinical trial documentation pipeline. By facilitating automated and cohesive document creation, embodiments of the document generation system aim to become a central tool in the clinical trial field, promising a streamlined approach to fulfilling the varied documentation requisites that are important in contemporary clinical trial procedures.

In one embodiment, documents are generated by receiving clinical protocol documents and client template documents as input to a computer having a document management system, a data extraction model, a language model learning model, at least one knowledge database, and a document output processor. The computer maps the clinical protocol documents and client template documents to the document management system and then extracts vital information from the clinical protocol documents using the data extraction model. The computer then verifies and enriches the vital information using the language learning model operating on the vital information using data from the at least one knowledge database, the language learning model producing enriched data. The enriched data is then transformed to natural language using the language model learning model. A document output processor then generated clinical documents using the enriched data. This embodiment is able to apply the equivalent of vast science knowledge and experience to automatically generate accurate and complete, yet readable and understandable clinical documents.

The present invention together with the above and other advantages may best be understood from the following detailed description of the embodiments of the invention illustrated in the drawings, wherein:

### DRAWINGS

FIG. 1 is a flow chart illustrating high-level operation of an automated document generation system consistent with embodiments of the invention;

FIG. 2 is a flow chart illustrating the language processing step of FIG. 1;

FIG. 3 is a diagram of a portion of an example Language Model Learning fact graph consistent with embodiments of the invention;

FIG. 4 is a diagram of a portion of an example Symbolic AI fact graph consistent with embodiments of the invention;

FIG. 5 is a flow chart illustrating the process of filling placeholders consistent with embodiments of the invention;

FIG. 6 is a block diagram of a computer system for automatically generating clinical documents consistent with embodiments of the invention; and,

FIG. 7 is a flow chart illustrating the process of redaction consistent with embodiments of the invention.

### DESCRIPTION

Embodiments of automated document generation systems and methods operate to generate patient-facing documents and other documents needed in clinical trials. The systems and methods enable understandable and expedited generation of the documents. At least one embodiment includes the integration of AI models, a biochemistry-oriented knowledge base, and Language Model Learning (LLM) mechanisms. Embodiments of the document generation system are capable of quickly producing documents that balance technical correctness with readability, having the benefits of substantially enhancing the productivity of clinical trial initiation and management processes. Embodiments of the document generation system enable the application of vast knowledge and expertise in science and medicine and biochemistry to produce comprehensive and accurate yet also understandable writing. In addition, the embodiments of the document generation system reduce errors and omissions.

It is understood that one or more of the below methods, or aspects thereof, may be executed by at least one control unit. The term “control unit” may refer to a hardware device that includes a memory and a processor. The memory is configured to store program instructions, and the processor is specifically programmed to execute the program instructions to perform one or more processes which are described further below. Moreover, it is understood that the below methods may be executed by an apparatus comprising the control unit in conjunction with one or more other components, as would be appreciated by a person of ordinary skill in the art. Furthermore, the control unit of the present disclosure may be embodied as non-transitory computer readable media containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed throughout a computer network so that the program instructions are stored and executed in a distributed fashion.

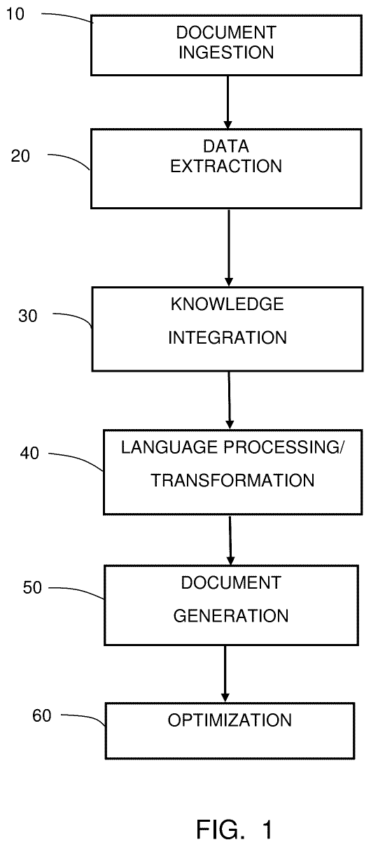

FIG. 1 is a flow chart illustrating the high-level operation of one embodiment of the automated document generation.

At step 10, clinical protocols are taken as input to the automated document generation system. This is also referred to as document ingestion. The system is trained to take in documents having a variety of formats. This enables the automated document generation system to easily integrate with existing databases and document systems.

Also at this step, the automated document generator accepts as input client templates related to particular clinical trials. The client templates are mapped inside a generic document management system to facilitate the precise alignment of the destination document (e.g., an informed consent form) with the source document (e.g., a protocol), ensuring that all placeholders in the destination document will be adequately populated from the source document. Examples of document management systems that may be used in embodiments of the present automated document generator include Microsoft Sharepoint, Google Drive, Dropbox or Box.com. Other document management systems, including open source systems, are possible within the scope of the present invention.

The ability to ingest various formats of documents operates to initialize the process of creating patient-facing documents by extracting required data from source documents. The present embodiment's capability of integrating and analyzing a range of document formats enables generally seamless initiation of the automated document creation process.

At step 20, the document generation system identifies vital information in the clinical protocols, that is, information related to understanding the clinical trial and important for informed consent. This step, which is also referred to as the data extraction step, serves as a foundation for generating accurate and comprehensive patient-facing documents.

In this step, the system uses advanced artificial intelligence (AI) models to identify and extract pertinent information from the ingested documents. The AI models work synergistically with the integrated knowledge base and Large Language Model (LLM) Learning technologies to optimize the data extraction process, ensuring a swift and efficient retrieval of necessary data components to be used in the subsequent stages of document creation. Models appropriate for use in this phase are adept at identifying, extracting, and categorizing pertinent information from client documents and other AI training documents. Industry-specific knowledge bases may also be used to train these models. With regard to documents that may be used in data extraction, non-structured text in clinical trial documents is appropriate for data extraction. This non-structure may exist in different layouts, such as paragraphs, headline-subheadline architecture, tables and more. In essence, the system has the ability to differentiate between these different layouts and still make sense out of it, pin point where the placeholders are and how the document needs to be generated. This approach may differ for different types of clinical trial documents in alternative embodiments.

Examples of AI models suitable for use by this system for data extraction include:

Convolutional Neural Networks (CNNs): Traditionally used for image processing, CNNs can also be applied for document analysis, especially when the structure of the document is a factor.

Recurrent Neural Networks (RNNs) and Long Short-Term Memory (LSTM) Networks: These models are especially suitable for sequential data, making them good choices for processing and understanding the structure of textual documents.

Transformers and Attention Mechanisms: Models like BERT (Bidirectional Encoder Representations from Transformers) and its variants can be effective for understanding context in textual documents. They can extract relevant information by paying “attention” to specific parts of the text based on context.

Named Entity Recognition (NER) Models: These models are designed to identify and categorize specific entities in text, such as medical terminologies, drug names, or specific procedures.

Topic Modeling Algorithms: Algorithms like Latent Dirichlet Allocation (LDA) can be used to understand the main topics in a document, providing a summarized view of its content.

Regular Expression Matching: For structured documents with known patterns, regular expression matching can be an efficient method to extract specific data points.

Word Embeddings: Models like Word2Vec or FastText can be used to understand semantic similarities between words in the documents and help in extracting contextually relevant information.

Rule-Based Systems: In scenarios where certain terms or patterns are predefined and consistently present across documents, rule-based extraction mechanisms can be used.

Graph Neural Networks (GNNs): These are often useful where the data has a relational structure, allowing the system to understand and extract relationships and entities more effectively.

In one embodiment, a specific AI model is used in this step. In an alternate arrangement, a combination of models is used in response to the nature and format of the clinical trial protocol documents, as well as in response to the specific requirements of the data extraction process.

At step 30, referred to as the knowledge integration step, the extracted data is cross-verified and enriched using at least one knowledge base such as a database specialized in biochemistry. This step provides depth and reliability to the extracted data. Example knowledge bases used in this step are CDC EveryDay Words and Wikipedia Biochemistry data. Example Language learning models used in this phase include Named Entity Recognition (NER), large language models (LLM), Regular Expression Matching and Word Embeddings. In this step, extracted data from clinical trial documents is checked against a science database appropriate for the particular clinical trial, for example, a biochemistry knowledge base, ensuring accuracy and alignment with known scientific facts.

Then, the data is enriched. This operation includes filling null values where missing data are populated using the knowledge base (extracted from sample clinical documents and verified with domain knowledge and templates). Data enrichment further includes adding features where relevant data, not in the original document but present in the knowledge base, are incorporated. Data enrichment still further includes identifying and adding synonyms, that is, alternate names or synonyms for terms or drugs which are added for clarity. Depth is additional detailed information that is added to the extracted data. For example, a mentioned drug might be supplemented with its mechanism of action. Context is background or related information such as the frequency of a drug's side effect.

This step enriches the data pool available for document generation, adding depth and context to the extracted information, thereby contributing to the creation of technically sound patient-facing documents. In short, the knowledge integration step refines and expands the extracted data, preparing it for further processing. The knowledge integration phase is important as it adds depth and context to the data extracted, enhancing the quality of the output documents. This step increases the soundness of the generated patient-facing documents generally lacking in human-created documents.

Performing knowledge integration using a specialized knowledge base specifically designed for deeper training in the biochemistry niche facilitates the generation of technically profound and nuanced documents, fulfilling a gap not generally addressed by conventional AI document writers. The present embodiment further includes further Advanced Language Model Learning, a harmonious component that cooperates with AI and the knowledge base, enhancing the overall document creation process. The present embodiment ensures the formulation of coherent and comprehensible text, making complex data accessible to a broader audience.

At step 40, the language processing step, the system transforms complex, technical data into natural language, making the patient-facing documents understandable to a broader audience. Using Language Model Learning, the enriched data are translated into coherent and easily understandable text, forming the backbone of the content that will populate the destination document. The language processing step translates the enriched data into coherent, easily understandable text. With this operation, the system is able to convert the technical data into a format that is accessible to a broader audience, making it more effective and efficient. The presence of this step delivers the benefit of providing clear and comprehensible patient-facing documents.

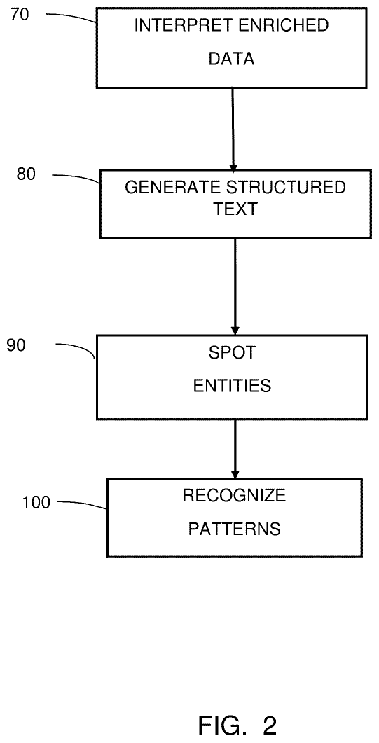

In the language processing step, the system uses Language Model Learning, along with other language processing models like Named Entity Recognition (NER) and Regular Expression (Regex), to transform the enriched and verified data into coherent text. This step is illustrated in detail in FIG. 2. This entails:

Semantic Understanding (step 70): The system interprets the enriched data, ensuring that the content is translated into natural language while maintaining its original meaning and context.

Structural Formation (step 80): Using LLM and complementary models, the generated text is given proper structure, grammar, and flow, making it clear and readable.

Entity Identification (step 90): The NER models, for example, are used to spot and categorize specific entities in the text. Specific entities include text elements such as drug names, medical conditions, or specific procedures.

Pattern Recognition (step 100): Regex, for example, can be used to assist in identifying and extracting information based on known patterns, ensuring consistent formatting and data representation.

In this embodiment, by combining Language Model Learning with NER and Regex, the Language Processing step efficiently translates structured data into a well-formulated narrative, preparing it for the final document generation.

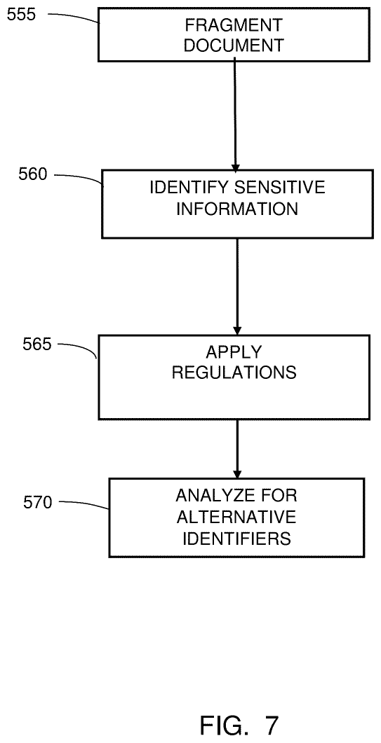

Returning to FIG. 1, at step 50, the system generates documents including patient-facing documents using a document output processor. The system generates patient-facing documents that are in compliance with existing legal and ethical standards, thereby safeguarding trial participants' rights and well-being. In one embodiment, the system performs the operations of generating documents using the source documents and templates stored within it. In another embodiment, the automated document generator system includes redaction knowledge. Some clinical documents such as clinical trial agreements (CTA) have information redaction requirements such as the personal identifying information of clinical trial participants. The models in this embodiment include redaction rules and training for information to be redacted. An embodiment of redaction is described below with regard to FIG. 7. In a further alternative embodiment, publishing rules are included and documents following publishing guidelines are generated. Publishing requirements are often detailed and strict and typically difficult to achieve. Automated generation of document in compliance with publishing requirements saves time and considerable expense. Generating documents includes calculating the values for placeholders as described below.

In another embodiment, the system interacts with a dedicated document management site, such as the document management site owned and maintained by an organization running or sponsoring a clinical trial. The system takes a mapped template and source documents from the dedicated document management site, calculates the placeholders to produce a final destination document.

The document generation process in this step includes receiving template documents configured to serve as a foundation for the desired destination documents. The software system maps a sample input file into the various placeholders in the template document. The mapping enables the document generation system to recognize the format and expected content area within the received template. The mapping process is also referred to as annotating the document. The annotations act as markers guiding the system to know where particular pieces of information from an input file should be placed in a destination document.

At step 60, referred to as the feedback and optimization step, the automated document generation system facilitates continuous improvement, enhancing its efficiency and output quality with each iteration. This is step is considered optional and not integral to the operation of the automated document generation system. In this step, the system learns from user feedback and previous interactions to continuously enhance the output quality over time. This continuous improvement cycle enables the scalability of automated document generation system by allowing the same process to be replicated for creating destination documents for additional clinical studies. While this step is designed to enhance the quality and efficiency of the system over time, technically, the invention could still function without it. This step, though not integral to the basic function of embodiments of the invention, contributes to its progressive improvement and adaptation, making it a valuable component for long-term efficiency and success.

Another example embodiment of the automated document generation system includes a generative-symbolic AI model which is trained to generate medical documents such as ICF, CTR, PLS, and CSR. A generative-symbolic AI model is also known as logic-based AI and refers to a part of AI involving high-level symbolic, i.e. human-readable, representations of problems.

ICF is a document framework of the World Health Organization. ICF stands for “International Classification of Functioning, Disability and Health”. ICF is the World Health Organization framework for measuring health and disability at both the individual and population levels. It is a classification of health and health-related elements including environmental factors. CTR is an EU regulation. Clinical Trials Regulation (CTR) is pharmaceutical legislation based on the public policies of offering a favorable environment for carrying out clinical research on a large scale with high standards of public transparency and safety for the clinical trial participants. PLS are plain language summaries. PLS are summaries of scientific research written in language that is easy to read and understand. CSR stands for Clinical Study Report. In medicine, a clinical study report (CSR) on a clinical trial is a document, typically very long, providing detail about the methods and results of a trial and including information about efficacy and safety.

The medical document templates for clinical trials such as the ICF, CTR, PLS and CSR documents, contain placeholders to be filled with information regarding the participants of the clinical trial and other information regarding the clinical trial. The placeholders are described below in conjunction with an example document template. The placeholders are of various types and the model operating inside the clinical document generation system follows instructions and applies readability and text formatting operations.

There are a number of types of placeholders: simple placeholders, logical placeholders, and readability and text formatting placeholders. In the example document below, the underlined sections are the placeholders. The placeholders will be identified and described in detail after the document.

### Example Document Template

1. This is a research study and your participation is voluntary.
2. This study drug is intended to treat patients with <disease > (brief lay description of disease).
3. The study drug is “investigational”. This means that it has not been approved by the United States Food and Drug Administration (FDA).
4. This is a <type of study (e.g. first in-human, Phase 1, Phase 2)> study. This means the study drug has never been given to humans before. The list of known risks/discomforts is listed later in this informed consent.
5. There will be up to <XX> participants total in this study.
6. <Brief lay descriptions of study design including purpose/objectives>:

If you participate in <lay terminology for SAD portion of the study>, the study should take about <XX (applicable time unit)> of your time and you will be given a clay terminology for route of administration of study drug> of <study drug ID> or placebo (contains no active ingredient) on Day 1.

If you participate in <lay terminology for repeat-dose portion of the study>, the study would take about <XX applicable time unit> of your time and you will be given a <lay terminology for route of administration of study drug> of <study drug ID> or placebo <lay terminology for dosing regimen/duration>.

During the <lay terminology for repeat-dose portion of study>.

X cohorts of X participants will be given <lay terminology for route of administration of study drug> of study drug ID or placebo <lay terminology for dosing regimen/duration>. The dose given <lay terminology for choice of dose for repeat-dose portion of study based on data from SAD portion of study>.

During the <lay terminology for SAD portion of study>:

Up to X cohorts (or groups) of X participants will be given <lay terminology for route of administration of study drug> of study drug ID or placebo. The first cohort will receive the starting dose, and later cohorts will be given higher and higher dose levels until a safe limit is found. The starting dose will begin at X mg. This is XXXX-fold lower than the highest dose tested in animals.

If you qualify for the study, you will return to the clinic within XX days for the Study Period.

Vitamins or herbs you have taken since your last visit.

<Bullet list of lay terminology of all assessments required during Check-in/Day 1>.

### End Example Document

In the document above, the following placeholders are examples of simple placeholders: “<disease>”, “<study drug ID>”, and “<type of study (first in human, Phase 1, Phase 2)”. The following placeholders are examples of logical placeholders: “There will be up to XX participants total in this study”, “In repeat dose portion of the study, X cohorts of X participants”, “In single ascending dose portion of the study, X cohorts (or groups) of X participants”, and “You will return to the clinic within XX days for the Study Period.” The following placeholders are readability placeholders: “<brief lay description of disease> (combination of simple and readability)”, “<lay terminology for route of administration of study drug>”, “<lay terminology for repeat-dose portion of study>”, “<lay terminology for SAD portion of study>”. The following placeholder is a text formatting placeholder: “<Bullet list of lay terminology of all assessments required during Check-in/Day 1>”.

The generative-symbolic AI model makes use of source documents and instructions in prompt files to create a facts graph. A prompt file is a set of instructions stored in the document generation system. The instructions are for managing placeholders, logical resolutions and output formatting to enable consistency in resolving variables and enable generated documents that adhere to the input documents. The Language Learning Models manager also uses the prompt files. Subsequently, the AI model, depending on the type of placeholder it is working on, maps the placeholder to the facts graph to simply extract its value or compute its value from more than one fact.

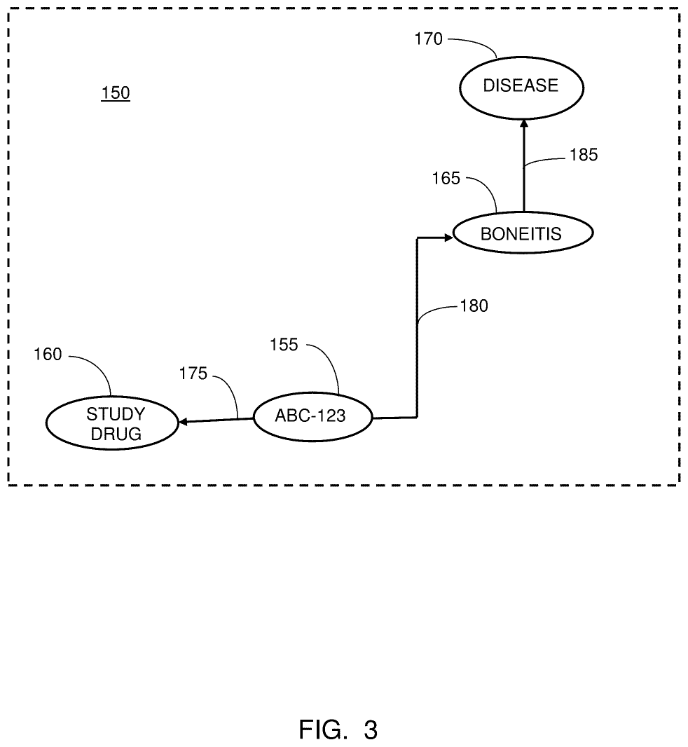

The Language Model Learning creates a graph of facts, also referred to as a “fact graph” from source documents and instructions in prompt files. FIG. 3 shows a small section of an example Language Model Learning fact graph 150. A fact graph includes nodes of facts suitable for insertion into a placeholder in a document with connections between the nodes that provide context for the facts in the nodes. In FIG. 3, the fact graph 150 includes the nodes ABC-123 155, study drug 160, boneitis 165 and disease 170. The nodes 155, 160, 165, 170 are connected with connectors 175, 180, 185 providing information about the relationship between the connected nodes. ABC-123 155 and study drug 160 have a definition connector 175 between them. ABC-123 155 and boneitis 165 have an interaction connector 180 between them. Boneitis is identified as a disease by the connector 185 between its' node 165 and the disease node 170. This is merely a small example of a fact graph. Fact graphs are typically far larger.

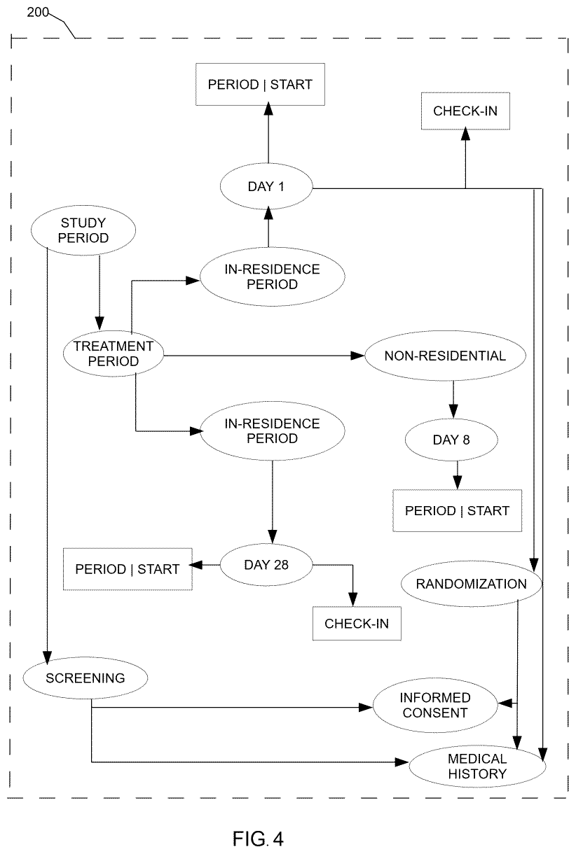

FIG. 4 is a portion of an example graph of facts 200 created by Symbolic AI algorithms in accordance with embodiments of the automated document generator. Symbolic AI algorithms mainly act on structured elements in the documents like tables, key-value pairs etc. In this example, a table of study procedures, screening tests, and daily records of the procedures is provided to the Symbolic AI. The table was created from the source documents provided to the system including the documents for the particular clinical trial for which documents are to be generated. The Symbolic AI creates a graph of facts including a portion of the facts graph 200 shown in FIG. 4.

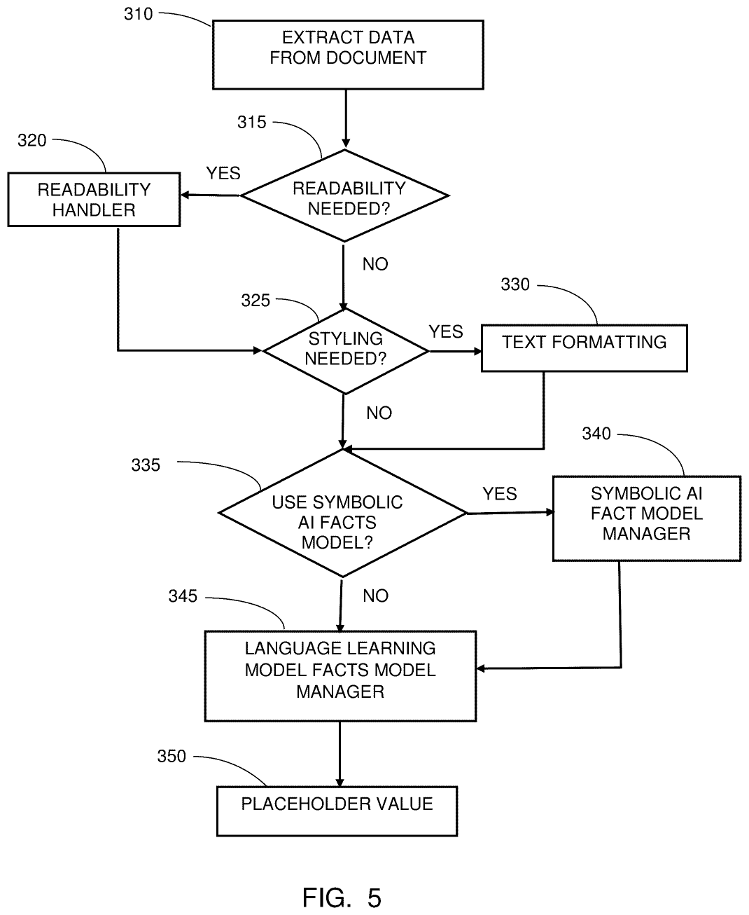

FIG. 5 is a flow chart illustrating the process of filling placeholders which is part of the document generation process.

At step 310, the system takes a placeholder element, also referred to as a “prompt” from the document as input. There are a few types of placeholders: simple placeholders, logical placeholders, and readability and text formatting placeholders.

At step 315, the system decides if the placeholder has a readability component. If yes, then the system proceeds to step 320, and invokes the readability handler. The readability handler infuses readability instructions and a definition dictionary of complex medical terms into the prompt to increase readability. Further, the readability tests the generated text for the prompt using the following tests: Flesch-Kincaid grade level, SMOG grade, and Gunning Fog Index. If no readability is needed, the system proceeds to the next decision step.

At step 325, the system decides if the placeholder has a style component. If yes, then the system proceeds to step 330, text formatting. If no, then the system proceeds step 335.

At step 335, the system determines if the Symbolic AI Model is needed. If yes, the system uses, step 340, the Symbolic AI Fact model manager to add a value to the placeholder element. The Symbolic AI Fact model manager accesses a fact graph such as the one in FIG. 4. If the Symbolic AI Model is not needed, the system continues to step 345.

At step 345, the system applies the Language Learning Model Facts model manager. The Language Learning Model Facts model manager uses a fact graph such as the one on FIG. 3 to determine a value for the placeholder. The placeholder element may include specific instructions to assist the AI in logic and reasoning in both this step and in step 340.

At step 350, the system outputs a placeholder value to be used in a generated document.

FIG. 7 is a flow chart of an embodiment of the redaction method included in some embodiments of the automated document generator. The redaction method identifies and protects sensitive information within medical documents. Sensitive information may also be defined as “private” or “protected” information.

At step 555, the system fragments the document. In document fragmentation, the system analyzes the document structure, typically focusing on a table of contents. The information is used to fragment the document into distinct sections thereby enabling a targeted and context-aware redaction process.

At step 560, the system uses Named Entity Recognition (NER) and Large Language Models (LLM) and regular expression (Regex) patterns to identify sensitive information in the document. NER detects potentially sensitive information across various contexts within a document. NER is a natural language processing technique that identifies and classifies named entities in text into predefined categories such as person names, organizations, locations, medical codes. LLMs are used to understand the context and content of the text more deeply, identifying sensitive information that might not be caught by simpler pattern-matching techniques. The system applies Regex patterns to identify specific text formats such as phone numbers, email addresses.

At step 565, the system analyzes the documents according to at least one set of regulations. The regulations are for example, the rules of the European Medicines Agency (EMA). The EMA rules include guidelines for what types of information should be redacted in medical documents. The information to be redacted includes patient identifiers, proprietary drug information and confidential research data.

At step 570, the system analyzes the documents for alternative identifiers. Classic identifiers include identifying information such as name, patient number, and Social Security number. In general, a classic identifier is any piece of information that is meant to identify the person as an individual. There are instances where redaction of classic identifiers is not enough to protect confidentiality. It is sometimes possible to identify a person based on a non-classic identifier or by using a combination of non-classic identifiers. That is, in some cases, a piece of information or a combination of pieces of information may be identifying. For example, in a case having clinical, geographic and demographic data, e.g. a cancer site, a combination of information such as county and race may be identifying, particularly in regions of low population density. The system at this step applies a methodology for finding non-classical identifiers.

The context-aware processing capability of the automated document generator enables intelligent application of the redaction steps above. By fragmenting the document based on its table of contents, different sections can be subjected to appropriate redaction steps. Once sensitive information is identified, the system applies redaction, for example, by removing the identified text and replacing it with black boxes. This process ensures that the redacted information cannot be recovered from the PDF file, maintaining the confidentiality and integrity of the sensitive data.

Confabulations (also known as “hallucinations”) can occur when there has been insufficient or ineffective training data, poorly written instructions, idioms or slang that the system cannot comprehend, or model overfitting (where the AI is memorizing answers because of limited training). Embodiments of the invention address this issue in one or more of several ways. First, the Language Learning model (an example of which is shown in FIG. 3) is trained on multiple libraries of clinical document source material. In an alternate arrangement, an embodiment of the system uses both clean copies of documents and templates to streamline the instruction processing. In a further alternate arrangement, every document type is trained on multiple sets of documents, including publicly available documents and templates, to ensure effective training.

Alternative embodiments include alternative elements described below.

Document Ingestion: In this alternative embodiment, the document ingestion process incorporates more advanced file recognition and parsing systems to handle a wider variety of document formats and data types without altering the basic function of ingesting necessary data for the creation of patient-facing documents.

Data Extraction: In this alternative embodiment, the data extraction step uses more sophisticated AI algorithms capable of identifying nuanced patterns or trends in the data. These algorithms are for example deeper learning techniques for a more intricate extraction process without changing the fundamental role of data extraction.

Knowledge Integration: In this alternate embodiment, while the core concept of integrating a knowledge base remains constant, the extent and depth of the knowledge base is expanded. Incorporating real-time updates or access to a more extensive array of databases makes the integration more dynamic and adaptable without altering the basic principle of enriching data with necessary information.

Language Processing: In this alternate embodiment, the language processing module integrates advanced natural language processing (NLP) techniques or linguistic models to enhance the readability and comprehensibility of the output texts. This involves improved handling of technical jargon and complex concepts without straying from the primary goal of translating data into understandable text.

Document Generation: In this alternate embodiment, the document generation step is augmented by integrating dynamic template generation. This enables customization of document layouts and formats, potentially incorporating multimedia elements or interactive components without changing the central objective of generating patient-facing documents adhering to necessary guidelines.

Feedback and Optimization: In this alternate embodiment, the feedback and optimization involve sophisticated analytical tools and metrics to gauge user satisfaction and document efficiency precisely. In an alternative arrangement, the feedback system incorporates AI-driven predictive analytics to foresee potential areas of improvement without changing the essential characteristic of enhancing the system's efficiency and output quality over time.

Alternate names for elements described above include the following:

Document Ingestion may be alternately known as an input module. This part can be referred to as the “input module”, which is responsible for accepting and recognizing various document formats, essentially serving as the starting point for the document processing pipeline.

Data Extraction may be alternately known as an information retrieval system. This part can be dubbed the “information retrieval system”, where essential data are identified and extracted from the source documents, laying the foundation for the automated creation of patient-facing documents.

Knowledge Integration is alternately known as the data enrichment layer. The “data enrichment layer” encapsulates the knowledge integration step generically, indicating the role of this layer in adding depth and context to the initially extracted data, enhancing the eventual output.

Language Processing may be alternately known as the Natural Language Processing Unit. This part serves as the “natural language processing unit”, where complex data is translated into natural, comprehensible language, facilitating clearer communication in the resultant documents.

Document Generation may be alternately known as the output generation system.

The “output generation system” is a generic term that represents the phase where the processed data are compiled into the final document, adhering to necessary regulatory and ethical standards.

Feedback and Optimization may be alternately known as the Adaptive Learning Module. This can be referred to as the “adaptive learning module”, illustrating the system's ability to learn and evolve over time based on feedback and prior interactions to enhance output quality progressively.

While each component of the embodiments described above play important roles in the overall functioning, there could be potential avenues for altering the functionalities, combining steps, or even streamlining the process by eliminating certain aspects to create further alternative embodiments, as follows:

### Changing Functions

Feedback and Optimization: This step could potentially be changed to incorporate more proactive analytical tools, predicting areas of improvement based on current trends and feedback, thereby transforming it into a more predictive component rather than being merely reactive.

### Combining Steps

Data Extraction and Knowledge Integration: These steps could be combined to form a singular, more powerful data processing unit where information extraction and enrichment occur simultaneously, utilizing an integrated AI system that can extract data while cross-verifying and enriching it in real time.

Language Processing and Document Generation: These steps might be merged into a comprehensive document creation module where the transition from data translation to document generation is seamless, enabling the generation of documents in real time as the data is processed and translated into natural language.

### Eliminating Steps

While theoretically possible, eliminating any of the existing steps might significantly impact the system's efficacy and output quality. However, in a more streamlined version of the invention:

Feedback and Optimization: This step might be seen as optional, especially in the initial stages of deployment where the focus is more on establishing a robust base functionality. As the system matures, this step can be reintroduced to further hone and optimize the process based on user feedback and experiences.

To enhance the functionality and efficiency of the automated document generator, several features or modules could be incorporated. Here are a few possibilities within the scope of the present invention:

Multi-Lingual Support: Integration of a multi-lingual support system that can automatically translate the generated patient-facing documents into various languages, enhancing the software's accessibility and reach globally.

Advanced Security Protocols: Incorporation of advanced security protocols to safeguard sensitive information, ensuring data privacy and compliance with global data protection regulations.

Integrated Compliance Checker: A module that continuously updates and checks the generated documents against the latest industry regulations and guidelines, ensuring that they are always compliant, reducing the risk of non-compliance penalties.

Customizable Templates: A feature that provides users with the ability to create and customize document templates, allowing for more personalized and industry-specific document generation, enhancing user experience and output relevance.

Machine Learning-Driven Quality Control: Integration of a machine learning-driven quality control system that can automatically identify and correct errors or inconsistencies in the generated documents, enhancing the accuracy and reliability of the output.

Blockchain Integration for Document Verification: Incorporation of blockchain technology to create immutable records of the generated documents, providing a secure and verifiable chain of custody for each document, enhancing trust and transparency in the document creation and management process.

User Training Module: A module that offers training and tutorials for users to efficiently use the software, understand its functionalities fully, and leverage its features to maximize productivity.

Feedback Loop with Continuous Learning: Enhancing the feedback and optimization step with a more dynamic continuous learning algorithm that adapts and learns from every interaction, constantly evolving and improving the system's output quality.

Analytics and Reporting: An analytics and reporting module that provides insights into the usage patterns, efficiency metrics, and other relevant data, aiding in informed decision-making and continuous improvement of the system.

By integrating these additional features, embodiments of automated document generator could offer more robust, secure, and user-friendly functionalities, further streamlining the document creation process and enhancing the overall efficiency and effectiveness.

Alternative uses for embodiments of the automated document generator are as follows: automations for medical writers for day-to-day writing tasks; automating writing including standard outputs and formulated statements the medical writer can interrogate, regardless of document type; use to spot and develop language describing common trends; use to spot and correct common formatting issues; pull in tables from source files including .rtf and .pdf files; help with document quality control; use to assess demographics and level of applicability of results in different subpopulations; selection of language from a lexicon of terms for a given endpoint; chatbot user can interact with within Word that leverages existing Q&A topics and curated Viva Topics and SharePoint lists (overall global questions that can be answered if there is no existing response); automate the creation of timelines and other Project Management tools for a given document type; make suggestions on conserving time if a deadline or assumption changes; and a wizard to bring the user through the creation and modifications.

In further alternate embodiments, the automated document generator is compatible with a plurality of document management systems to enable a wide range of usability.

In further alternate embodiments, to ensure the security and confidentiality of the data, the system uses encryption tools to safeguard the information stored and transmitted within the system.

In a further alternative embodiment, the automated document generator is implemented in a cloud computing environment. This has the benefit of enabling scalability and accessibility. It also allows for better management of resources and enables real-time updates without disrupting the user experience.

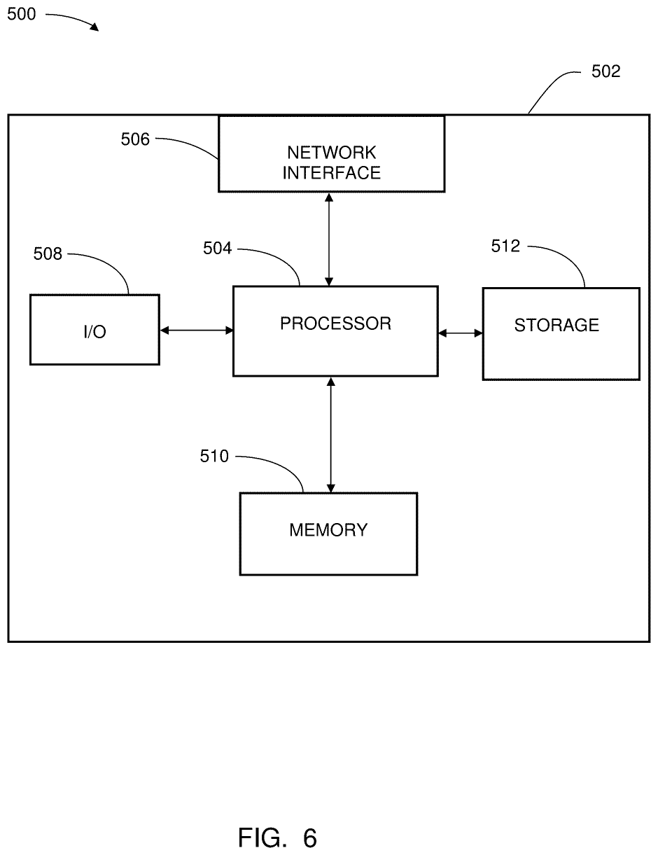

FIG. 6 is a high-level block diagram 500 of an example computer that may be used to implement systems and methods described herein. Computer 502 includes a processor 504 operatively coupled to a data storage device 512 and a memory 510. Processor 504 controls the overall operation of computer 502 by executing computer program instructions that define such operations. The computer program instructions, AI models and fact graphs may be stored in data storage device 512, or other computer readable medium, and loaded into memory 510 when execution of the computer program instructions is desired. Thus, the method steps of FIGS. 1, 2 and 5 can be defined by the computer program instructions stored in memory 510 and/or data storage device 512 and controlled by processor 504 executing the computer program instructions. For example, the computer program instructions can be implemented as computer executable code programmed by one skilled in the art to perform the method steps of FIGS. 1, 2 and 5. Accordingly, by executing the computer program instructions, the processor 504 executes the method steps of FIGS. 1, 2 and 5. Computer 502 may also include one or more network interfaces 506 for communicating with other devices via a network. Computer 502 may also include one or more input/output devices 508 that enable user interaction with computer 502 (e.g., display, keyboard, mouse, speakers, buttons, etc.).

Processor 504 may include both general and special purpose microprocessors, and may be the sole processor or one of multiple processors of computer 502. Processor 504 may include one or more central processing units (CPUs), for example. Processor 504, data storage device 512, and/or memory 510 may include, be supplemented by, or incorporated in, one or more application-specific integrated circuits (ASICs) and/or one or more field programmable gate arrays (FPGAs).

Data storage device 512 and memory 510 each include a tangible non-transitory computer readable storage medium. Data storage device 512, and memory 510, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 508 may include peripherals, such as a printer, scanner, display screen, etc. For example, input/output devices 508 may include a display device such as a cathode ray tube (CRT) or liquid crystal display (LCD) monitor for displaying information to the user, a keyboard, and a pointing device such as a mouse or a trackball by which the user can provide input to computer 502.

Any or all of the systems and apparatus discussed herein may be implemented using one or more computers such as computer 502. One skilled in the art will recognize that an implementation of an actual computer or computer system may have other structures and may contain other components as well, and that FIG. 6 is a high-level representation of some of the components of such a computer for illustrative purposes.

It is to be understood that the above-identified embodiments are simply illustrative of the principles of the invention. Various and other modifications and changes may be made by those skilled in the art which will embody the principles of the invention and fall within the spirit and scope thereof.

We claim:

1. A method for automatically generating clinical documents, comprising:
receiving clinical protocol documents and client template documents as input to a computer having a document management system, a data extraction model, a language model learning model, at least one knowledge database, and a document output processor;
mapping the clinical protocol documents and client template documents to the document management system;
extracting vital information from the clinical protocol documents using the data extraction model;
verifying and enriching the vital information using the language learning model operating on the vital information using data from the at least one knowledge database, the language learning model producing enriched data;
transforming the enriched data to natural language using the language model learning model; and
generating clinical documents in the document output processor.
